SEQUENCE LISTING (24 sequences)

SEQ ID NO: 1 - DNA, 21 bp, Artificial Sequence
  Oligonucleotide for detection of wild type codon 12 of K-ras gene
gttggagctggtggcgtaggc

SEQ ID NO: 2 - DNA, 21 bp, Artificial Sequence
  Oligonucleotide for detection of a missense mutation in codon 12 of K-ras gene
agttggagcttgtggcgtagg

SEQ ID NO: 3 - DNA, 21 bp, Artificial Sequence
  Oligonucleotide for detection of a missense mutation in codon 12 of K-ras gene
agttggagctagtggcgtagg

SEQ ID NO: 4 - DNA, 21 bp, Artificial Sequence
  Oligonucleotide for detection of a missense mutation in codon 12 of K-ras gene
agttggagctcgtggcgtagg

SEQ ID NO: 5 - DNA, 21 bp, Artificial Sequence
  Oligonucleotide for detection of a missense mutation in codon 12 of K-ras gene
gttggagctgatggcgtaggc

SEQ ID NO: 6 - DNA, 21 bp, Artificial Sequence
  Oligonucleotide for detection of a missense mutation in codon 12 of K-ras gene
gttggagctgctggcgtaggc

SEQ ID NO: 7 - DNA, 21 bp, Artificial Sequence
  Oligonucleotide for detection of a missense mutation in codon 12 of K-ras gene
gttggagctgttggcgtaggc

SEQ ID NO: 8 - DNA, 21 bp, Artificial Sequence
  Oligonucleotide for detection of a missense mutation in codon 12 of K-ras gene
ttggagctggaggcgtaggca

SEQ ID NO: 9 - DNA, 21 bp, Artificial Sequence
  Oligonucleotide for detection of a missense mutation in codon 12 of K-ras gene
ttggagctgggggcgtaggca

SEQ ID NO: 10 - DNA, 21 bp, Artificial Sequence
  Oligonucleotide for detection of a missense mutation in codon 12 of K-ras gene
ttggagctggcggcgtaggca

SEQ ID NO: 11 - DNA, 21 bp, Artificial Sequence
  Oligonucleotide for detection of wild type codon 13 of K-ras gene
ggagctggtggcgtaggcaag

SEQ ID NO: 12 - DNA, 21 bp, Artificial Sequence
  Oligonucleotide for detection of a missense mutation in codon 13 of K-ras gene
tggagctggtcgcgtaggcaa

SEQ ID NO: 13 - DNA, 21 bp, Artificial Sequence
  Oligonucleotide for detection of a missense mutation in codon 13 of K-ras gene
tggagctggtagcgtaggcaa

SEQ ID NO: 14 - DNA, 21 bp, Artificial Sequence
  Oligonucleotide for detection of a missense mutation in codon 13 of K-ras gene
tggagctggttgcgtaggcaa

SEQ ID NO: 15 - DNA, 21 bp, Artificial Sequence
  Oligonucleotide for detection of a missense mutation in codon 13 of K-ras gene
ggagctggtgccgtaggcaag

SEQ ID NO: 16 - DNA, 21 bp, Artificial Sequence
  Oligonucleotide for detection of a missense mutation in codon 13 of K-ras gene
ggagctggtgacgtaggcaag

SEQ ID NO: 17 - DNA, 21 bp, Artificial Sequence
  Oligonucleotide for detection of a missense mutation in codon 13 of K-ras gene
ggagctggtgtcgtaggcaag

SEQ ID NO: 18 - DNA, 21 bp, Artificial Sequence
  Oligonucleotide for detection of a missense mutation in codon 13 of K-ras gene
gagctggtggtgtaggcaaga

SEQ ID NO: 19 - DNA, 21 bp, Artificial Sequence
  Oligonucleotide for detection of a missense mutation in codon 13 of K-ras gene
gagctggtggagtaggcaaga

SEQ ID NO: 20 - DNA, 21 bp, Artificial Sequence
  Oligonucleotide for detection of a missense mutation in codon 13 of K-ras gene
gagctggtggggtaggcaaga

SEQ ID NO: 21 - DNA, 24 bp, Artificial Sequence
  forward primer
ggcctgctgaaaatgactgaatat

SEQ ID NO: 22 - DNA, 27 bp, Artificial Sequence
  reverse primer
tgttggatcatattcgtccacaaaatg

SEQ ID NO: 23 - DNA, 21 bp, Artificial Sequence
  forward primer for sequencing
ggtggagtatttgatagtgta

SEQ ID NO: 24 - DNA, 22 bp, Artificial Sequence
  reverse primer for sequencing
ggtcctgcaccagtaatatgca

(19) United States
(12) Patent Application Publication
(10) Pub. No.: US 2007/0298419 A1
(43) Pub. Date: Dec. 27, 2007

(54) K-Ras Oligonucleotide Microarray and Method for Detecting K-Ras Mutations Employing the Same

(76) Inventors: Jae-Gahb Park, Seoul (KR); Il-Jin Kim, Seoul (KR); Hio-Chung Kang, Seoul (KR); Jae-Hyun Park, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 11/587,569

(22) PCT Filed: Nov. 22, 2004
(86) PCT No.: PCT/KR04/03023

§ 371 Date: Oct. 25, 2006

(30) Foreign Application Priority Data
Apr. 27, 2004 (KR) 10-2004-0028926

(51) Int. Cl. C12Q 1/68; C12M 1/00

(52) U.S. Cl. 435/6; 435/287.2

(57) ABSTRACT

Since the K-ras oligonucleotide microarray of the present invention can detect K-ras mutations by applying a competitive DNA hybridization method to the oligonucleotides spotted on a solid matrix different from the previously reported method for detecting a mutation, it makes the more precise analysis and can reduce experimental cost and time. Accordingly, the K-ras oligonucleotide microarray of the present invention can be used in studies to detect K-ras mutations and unravel the signal transduction mechanism and tumorigenesis related to K-ras gene. Further, since the microarray of the present invention can be applied to other genes having mutational hot spot regions such as K-ras, it has wide applicable range.

### FIELD OF THE INVENTION

The present invention relates to a K-ras oligonucleotide microarray for detecting mutations in the mutational hot spot regions of K-ras gene, a manufacturing process thereof and a method for detecting K-ras mutations employing the same.

### BACKGROUND OF THE INVENTION

K-ras is one of ras genes that undergo mutation in various cancers. The mutation of the K-ras gene at codons 12 and 13 takes part in tumorigenesis which leads to functional modification of p21-ras protein, a K-ras gene product, resulting in transferring excessive growth signals to a cell nuclei to stimulate cell growth and division. K-ras mutations are known to occur in roughly 90% of pancreatic cancer, 50% of colorectal cancer and 30% of non-small cell lung cancer and its mutation profile has revealed that about 85% of mutations occur at codons 12 and 13 (Samowitz W S, et al., Cancer Epidemiol. Biomarkers Prev. 9: 1193-1197, 2000). Therefore, identification of mutations of K-ras gene has been widely used as a useful tool in cancer diagnosis, e.g., pancreatic, colorectal and non-small cell lung cancers, and studies have suggested that it might be associated with some tumor phenotypes (Samowitz W S, et al., Cancer Epidemiol. Biomarkers Prev. 9: 1193-1197, 2000; Andreyev H J, et al., Br. J. Cancer 85: 692-696, 2001; and Brink M, et al., Carcinogenesis 24: 703-710, 2003). However, such studies usually required large number of samples to find out any meaningful link between K-ras mutation and specific clinical features (Andreyev H J, et al., Br. J. Cancer 85: 962-696, 2001), and there has been a demand in the field of epidemiology for a high-throughput technique, e.g., an oligonucleotide microarray can handle large samples with high accuracy and rapidity.

K-ras gene having mutational hot spots (codons 12 and 13) has been used as a target gene for testing new mutation detection techniques, e.g., a “DNA chip”. However, the previous studies employed specialized silicon devices or using complicated protocols to improve their system (Lopwz-Crapez E, et al., Clin. Chem. 47: 186-192, 2001; Prix L et al., Clin. Chem. 48: 428-435, 2002) which are not suitable for accurate and cost-effective evaluation of large samples.

Accordingly, the present inventors have developed a K-ras oligonucleotide microarray manufactured by fixing oligonucleotides on the surface of a solid matrix using an automatic microarrayer, the oligonucleotides being designed to detect various mutations at mutational hot spot regions of K-ras gene, and a new hybridization method, called Competitive DNA Hybridization (CDH), to increase both efficiency and capacity. The K-ras oligonucleotide microarray of the present invention can be used in studies to detect K-ras mutations and to unravel the signal transduction mechanism and tumorigenesis related to K-ras gene.

### SUMMARY OF THE INVENTION

Accordingly, an object of the present invention is to provide a K-ras oligonucleotide microarray which can be used as a fast and reliable genetic diagnostic device for studying the signal transduction mechanism and tumorigenesis related to K-ras gene as well as for detecting K-ras mutations.

In accordance with one aspect of the present invention, there is provided a K-ras oligonucleotide microarray for detecting K-ras mutations comprising a plurality of oligonucleotides fixed on the surface of a solid matrix, wherein the oligonucleotides are designed to detect missense mutation types at mutational hot spots of K-ras gene and comprise a wild-type having the nucleotide sequence of SEQ ID NO. 1 and missense mutation types having the nucleotide sequences of SEQ ID NOs: 2 to 10 at codon 12; and a wild-type having the nucleotide sequence of SEQ ID NO. 11 and missense mutation types having the nucleotide sequences of SEQ ID NOs: 12 to 20 at codon 13.

In accordance with still another aspect of the present invention, there is provided a method for detecting K-ras mutations employing same.

### BRIEF DESCRIPTION OF THE DRAWINGS

The above and other objects and features of the present invention will become apparent from the following description of the invention, when taken in conjunction with the accompanying drawings;

FIGS. 1a to 1e show the results of detecting K-ras mutations in the colon cancer tissue using the K-ras oligonucleotide microarray of the present invention with or without employing the CDH method;

| | |
|---|---|
| 1a: D231-control, | 1b: D231-CDH, |
| 1c: D281-control, | 1d: D281-CDH, |
| 1e: normal tissue of a cancer patient | |

### DETAILED DESCRIPTION OF THE INVENTION

The present invention provides a K-ras oligonucleotide microarray for detecting K-ras mutations, which comprises oligonucleotides fixed on the surface of a solid matrix using an automatic microarrayer, wherein the oligonucleotides are capable of detecting various mutations at mutational hot spot regions of K-ras gene.

First, the oligonucleotides are designed to detect all possible missense mutations at codons 12 and 13, mutational hot spots of K-ras gene.

Specifically, used for codon 1 are 9 types of substituted oligonucleotides obtained by replacing GGT (glycine) with TGT (cysteine), AGT (serine), CGT (arginine), GAT (aspartic acid), GCT (alanine), GTT (valine), GGA (glycine), GGG (glycine) and GGC (glycine), respectively. Used for codon 12 are 9 types of substituted oligonucleotides obtained by replacing GGC (glycine) with CGC (arginine), AGC (serine), TGC (cysteine), GCC (alanine), GAC (aspartic acid), GTC (valine), GGT (glycine), GGA (glycine) and GGG (glycine), respectively.

According to one aspect of the present invention, the K-ras oligonucleotide microarray of the present invention has 18 types of oligonucleotides spotted and fixed on the surface of a solid matrix, the oligonucleotides being capable of detecting various missense mutations at the 2 hot spot codons of K-ras gene. Nine oligonucleotides (M) are designed to cover all possible substitutions at each hot spot codon, and one oligonucleotide (W) for the wild type. Thus, a total of 18 oligonucleotides are designed to detect missense mutations for codons 12 and 13.

One wild type of oligonucleotide (W) is designed for each codon to be directly compared with mutation types and to cover both homozygous and heterozygous mutations. For example, 10 oligonucleotides are spotted for codon 12, one, a normal base sequence, and the rest (9), mutated base sequences. As a whole, 18 mutant oligonucleotides are designed for the 18 missense mutation types at the 2 hot spot codons, and 2 oligonucleotides, for the wild types and positive controls. Each oligonucleotide is spotted 4 times horizontally for increased accuracy of measured signals, which result in spotting a total of 80 oligonucleotides. Since the K-ras oligonucleotide microarray of the present invention has three sets of 80 oligonucleotides that are independently spotted on the surface of a solid matrix, it is capable of hybridizing with three different samples at the same time.

The present invention provides oligonucleotides which can be used to detect all possible mutations at the above mentioned mutational hot spot codons 12 and 13 of K-ras gene, which occur at a frequency of more than 85% in all cases examined. In addition, since the oligonucleotides used in the K-ras oligonucleotide microarray of the present invention are designed to detect all possible missense mutations at the 2 codons, it is capable of detecting any missense mutation at these codons which have not yet been discovered. Namely, as the oligonucleotides of the present invention are specifically designed to detect mutations at the hot spots of K-ras gene taking the gene characteristics into consideration, the K-ras oligonucleotide microarray of the present invention provides improved accuracy and efficiency in detecting K-ras gene mutation.

The K-ras oligonucleotide microarray of the present invention may be manufactured by fixing as many as 80 oligonucleotides on the surface of a solid matrix using an automatic microarrayer by a process comprising the steps of:

1) mixing each of the oligonucleotides in a micro spotting solution and distributing to a well plate;

2) spotting the oligonucleotide on the surface of a solid matrix using a microarrayer;

3) fixing the oligonucleotides on the solid matrix surface and washing;

4) denaturing the fixed oligonucleotides by soaking the solid matrix in 95° C. water, and then, treating the solid matrix with a sodium borohydride solution; and

5) washing and drying the solid matrix.

Each of the oligonucleotides used in step (1) preferably has a functional group that can be used to form a stable bond with the solid matrix surface. For example, each oligonucleotide may be linked with a 12 carbon spacer having a 5′ amino modification, e.g., H₂N—(CH₂)₁₂-oligonucleotide. This amine group undergoes Schiff's base reaction with an aldehyde group on the solid matrix to form a firm bond therebetween. The 12 carbon spacer serves to enhance the hybridization rate by facilitating the contact between the oligonucleotide and a fluorescent dye-labeled target DNA.

The micro spotting solution used in step (1) may contain suitable salts and polymers to facilitate the application of the oligonucleotides on the solid matrix.

The solid matrix used in step (2) may be made of glass, modified silicone, a plastic cassette, or a polymer such as polycarbonate or a gel thereof. The surface of a solid matrix may be coated with a chemical compound that can serve to bind the oligonucleotide to the matrix substrate. Preferable chemicals that can be used for such coating have functional groups such as aldehyde or epoxy groups. In one preferred embodiment, the present invention uses a slide glass coated with an aldehyde.

According to one embodiment of steps (1) and (2), a total of 80 oligonucleotides are arranged in a specified manner on a solid matrix using an automatic pin microarrayer. Each oligonucleotide spot is preferably of circular shape with a diameter ranging from 100 to 500 μm. A preferable example of the solid matrix is a 3.7 cm×7.6 cm slide glass, which can accommodate approximately 100 to 10,000 spots per chip. Preferably, a total of 80 oligonucleotide spots, each of 130 μm diameter, may be arranged in multiple columns and rows at intervals of 200 to 800 μm, preferably 300 μm.

In step (3), the oligonucleotides are fixed on the solid matrix surface by way of forming covalent bonds between the amine groups of the oligonucleotide and the aldehyde groups of the solid matrix via Schiff's base reaction. Free unreacted oligonucleotides are removed by washing the solid matrix with SDS (sodium dodecyl sulfate), SSC (standard saline citrate), SSPE (saline-sodium phosphate-EDTA), etc.

In step (4), the fixed oligonucleotides are denatured, and unreacted aldehyde groups remaining on the solid matrix are reduced and inactivated by sodium borohydride treatment.

The K-ras oligonucleotide microarray of the present invention manufactured by the above process may be advantageously used to detect gene mutation, and the method of the present invention is much simpler and more economical than any of the conventional gene mutation detection methods: It takes several days to months on the average when the presence of gene mutation is examined using such conventional methods as SSCP (single strand conformation polymorphism), PTT (protein truncation test), RFLP (restriction fragment length polymorphism), cloning, direct sequencing, etc. However, analysis of a DNA sample for K-ras gene mutation takes less than 10 to 11 hours when the K-ras oligonucleotide microarray of the present invention is employed. In addition, the K-ras oligonucleotide microarray of the present invention can be manufactured much more simply at a much less production cost than conventional chips. Once the required oligonucleotides are synthesized, it is possible to mass-produce the inventive slides. The amounts of reagents required when the K-ras oligonucleotide microarray of the present invention is used are far less than those required in any of the conventional methods.

The K-ras oligonucleotide microarray of the present invention is easy to manufacture using a pin microarrayer, while the existing Affymetrix oligonucleotide microarray must be prepared using a complicated and expense photolithography technique.

Further, it is possible with the K-ras oligonucleotide microarray of the present invention to purify and modify the oligonucleotides, in contrast to the case of Affymetrix oligonucleotide microarray which is prepared by directly synthesizing oligonucleotides on the surface of a solid matrix, during which it is not possible to purify or modify the oligonucleotides. In the K-ras oligonucleotide microarray of the present invention, it is capable of spotting the oligonuceltodies of high quality by purifying the oligonucleotides to increase their purity and easily modifying the oligonucleotides to reduce an experimental error. When regarding the fact that the quality of oligonucleotides to be spotted determines an overall reaction's accuracy in the oligonucleotide microarray, the K-ras oligonucleotide microarray of the present invention is capable of providing greater experimental accuracy than was possible before.

The present invention provides a method for detecting the K-ras mutation employing the K-ras oligonucleotide microarray, which comprises the steps of:

1) preparing a fluorescent dye-labeled DNA sample;

2) reacting the labeled DNA sample with oligonucleotide spots on the K-ras oligonucleotide microarray;

3) washing the reacted microarray to remove unbound sample DNA;

4) detecting the mode of hybridization of specific oligonucleotide spots using a fluorescence reader; and

5) examining the presence of gene mutation.

In step (1), a DNA sample is prepared by tagging a tumor specimen or a blood obtained from a subject patient with a fluorescent dye using PCR. The hybridization of the fluorescent dye-labeled DNA with certain oligonucleotide spots on the oligonucleotide microarray can be analyzed with a fluorescence reader using an appropriate software. Preferable fluorescent dyes include, but are not limited to, Cy5, Cy3, Alexa™ 594 fluor, Texas Red, Fluorescein and Lissamine.

In step (2), the florescent dye-labeled DNA sample prepared in step (1) is mixed with a hybridization solution and transferred to each of the oligonucleotide. At this time, the hybridization reaction may be carried out according to a competitive DNA hybridization (CDH) method which is based on the principle that mixed fluorescent dye-labeled DNAs each amplified from patients compete with each other in the hybridization reaction within the limited amount of spotted oligonucleotide.

For the CDH method, DNA samples are further labeled with two additional fluorescent dyes in addition to the fluorescent dye used in step (1). The additional fluorescent dye employable in this step includes all commercially available fluorescent dyes except the fluorescent dye used in step (1). In a preferred embodiment of the present invention, three fluorescent dyes, i.e., Cy3, Cy5 and Alexa™ 594 fluor, are introduced into DNA by amplifying each DNA sample with Cy3-, Cy5- and Alexa™ 594 fluor-labeled dNTPs, respectively. Each of Cy3-, Cy5- and Alexa™ 594 fluor-labeled DNA samples are mixed and hybridized together in one spotted region of microarray. The hybridization reaction is performed in a 45˜60° C. incubator saturated with water vapor for 3 hours.

Then, the microarray is washed to remove unbound sample DNA and dried (step 3), and the resulting fluorescence is analyzed with a fluorescence reader using an appropriate software (step 4). According to each fluorescent dye's excitation wavelength, hybridized microarray is scanned at wavelengths of 632.8 nm, 543.8 nm and 594 nm for Cys5, Cy3 and Alexa fluor, respectively (Lavmar L, et al., Nucleic Acids Res. 31: e129, 2003).

In step (5), setting a maximum value at 99% reliable range as a threshold value, any signal showing a fluorescence level higher than the threshold is regarded positive for the presence of mutation.

The K-ras oligonucleotide microarray of the present invention can be effectively used to diagnose such cancer as colorectal carcinomas, pancreatic cancer, non-small cell lung cancer, adenocarcinoma, squamous carcinoma, etc. Further, the K-ras oligonucleotide microarray of the present invention can be used as an effective diagnostic tool for the study of the signal transduction mechanism and tumorigenesis related to K-ras gene.

The advantages of the method for detecting the K-ras mutation employing the K-ras oligonucleotide microarray with the CDH method are as follows:

1) It can reduce signals from non-specific binding caused by small fragmented DNAs that might have homology with the spotted oligonucleotide and would compete in the hybridization.

2) Mutation analysis has been performed by calculating the ratio of mutation signal divided by wild-type (Kim I J, et al., Hardiman G ed. Microarrays methods and applications—nuts & bolts. Eagleville, DNA press, 249-272, 2003; Prix L, et al., Clin. Chem. 48: 428-435, 2002). Then the method of the present invention can regard it as a mutation having the ratio over threshold. Therefore, the greater ratio of them can be helpful to make the more precise analysis.

3) By mixing three samples labeled with three different fluorescent dyes, the method of the present invention can reduce experimental cost and time. In addition, the K-ras oligonucleotide microarray is designed to have three separated oligonucleotide sets, and thus, it is capable of investigating a total of 9 (3×3) samples per one microarray.

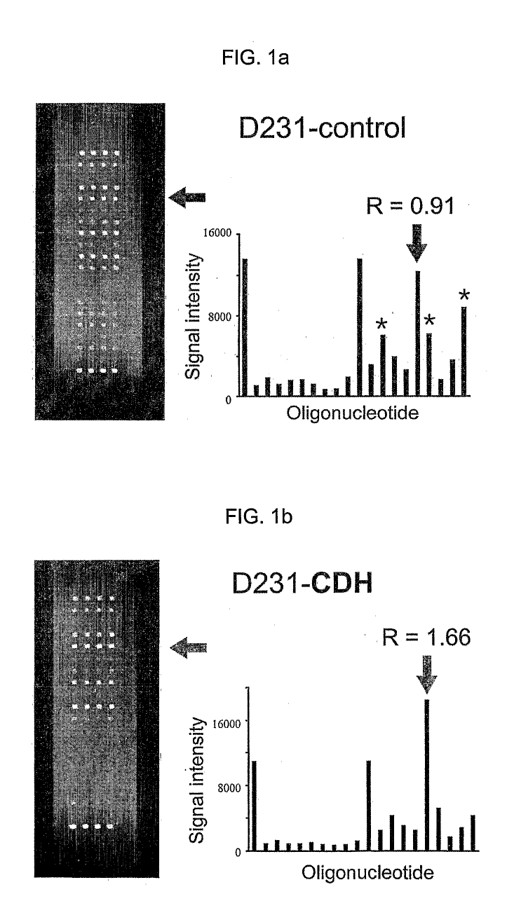

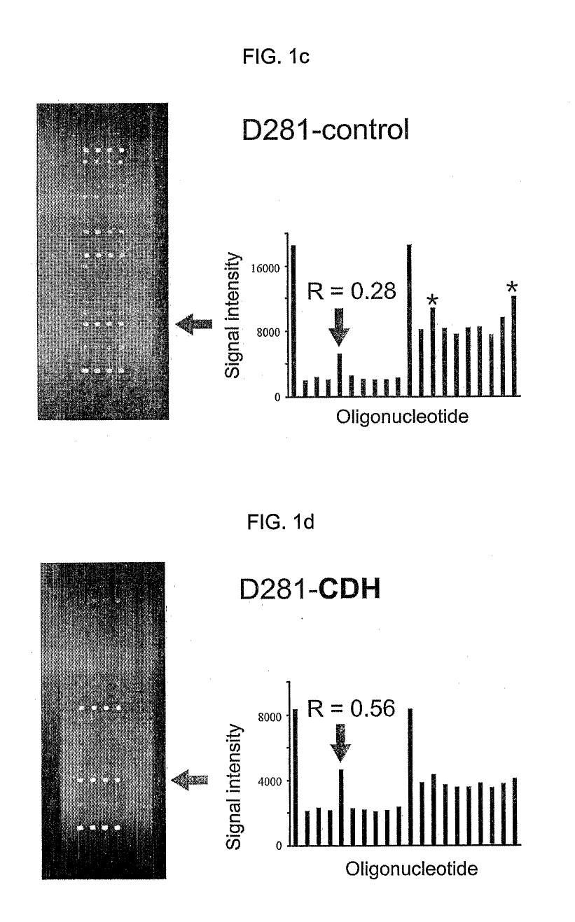

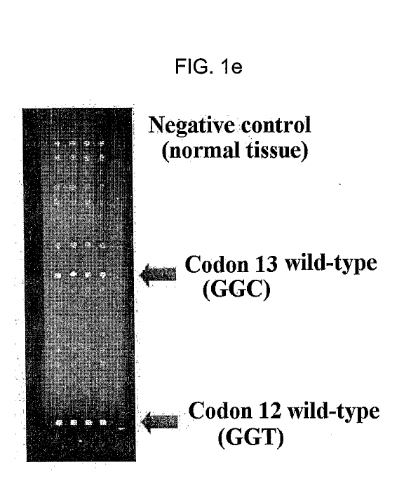

Although multiple fluorophores have been adopted in genotyping and DNA pooling focusing on parallel genotyping (Lovmar L, et al., Nucleic Acids Res. 31: e129, 2003; Hirschhorn J N, et al., Proc. Natl. Acad. Sci. USA 97: 12164-12169, 2000; Lindroos K, et al., Nucleic Acids Res. 30: e70, 2002), the present invention not only uses plural DNA samples labeled with multiple fluorescent dyes but also makes them compete with each other. In addition, the present invention intends to reduce the “cross-talk” problem, which is a phenomenon that the signal from one fluorophore is detected at more than one wavelength (Hirschhorn J N, et al., Proc. Natl. Acad. Sci. USA 97: 12164-12169, 2000). Since some fluorophores' spectrum of excitation and emission may overlap, their signals may be emitted when excited at other's wavelength. To prevent this phenomenon, the present invention has performed the CDH method using three different fluorescent dye-labeled dNTPs, Cy5-dCTP, Cy3-dCTP and Alexa fluor-dUTP having distinct spectra. As a result, the CDH method of the present invention shows a clearer image of the microarray by reducing signals from non-specific hybridization (see FIGS. 1a to 1e). It is also detected that two signals of wild-type (codons 12 and 13) are a little reduced. The reason is that fragmented wild-type DNA from each sample has to compete with each other for hybridization. But, mutant DNA, rarely found by same type in just three mixed samples, does not compete and conserved its original signal. As a result, when the sample has a mutation, the signal ratios between mutation and wild-type is increased from 0.91 to 1.66 (see FIGS. 1a and 1b) and from 0.28 to 0.56 (see FIGS. 1c and 1d).

204 Colorectal cancer patients were investigated for the presence of somatic K-ras mutation. As a result, a total of 50 mutations were identified in colorectal cancers ( 50/204, 24.5%) with the K-ras oligonucleotide microarray. Of these, 28 were from proximal colon cancers ( 28/103, 27.2%) and 22 were from distal colorectal cancers ( 22/101, 21.8%). The mutations detected above were classified into four types of missense mutation causing amino acid change in codons 12 and 13. The most common types of mutation were GGC (Gly)→GAC (Asp, 21/50) in codon 13. Others were changes from GGT (Gly) to GAT (Asp, 16/50), GTT (Val, 8/50), and TGT (Cys, 5/50).

Mutation results were 100% concordant with direct sequencing, showing neither false-positive nor false-negative. In order to investigate any significance between mutation profile and phenotype, statistical analyses were performed using the χ² or Fisher's exact test with SPSS software. α=0.05 was set as the significance level. It was found that GGT→GAT type is more prevalent in proximal colon cancer ( 13/28) that distal colon cancer ( 3/22, p=0.014) in concordance with previous reports (Samowitz W S, e al., Cancer Epidemiol. Biomarkers Prev. 9: 1193-1197, 2000; Brink M, et al., Carcinogenesis 24: 703-710, 2003). However, no significant relationship was detected between K-ras mutation and sex, age, tumor size, differentiation, and TNM stage.

In summary, it has been found that the results of K-ras oligonucleotide microarray exactly matched with conventional automatic sequencing, and the K-ras oligonucleotide microarray with the CDH method according to the present invention could increase efficiency in analyzing multiple samples. The K-ras oligonucleotide microarray is a sensitive, rapid, and high-throughput system thus may be suitable for the studies requiring large amount of samples, such as population-based study.

The following Examples and Test Examples are given for the purpose of illustration only, and are not intended to limit the scope of the invention.

### EXAMPLE 1

### Manufacture of K-ras Oligonucleotide Microarray

Eighteen oligonucleotides were designed to cover all possible substitutions at the two mutational hot spot codons of K-ras gene (codon 11 and 12), and two oligonucleotide for the wild-type. All oligonucleotides were 21 base pair and each mismatch sequence was located in the middle of oligonucleotides, as shown in Table 1. Oligonucleotides having missense mutation at one of the hot spot codons are: the oligonucleotides described in SEQ ID NOs. 2 to 10, at codon 12; and the oligonucleotides described in SEQ ID NOs. 12 to 20, at codon 13. The oligonucleotides described in SEQ ID NOs. 1 and 11 are wild types.

TABLE 1

| | | |
|---|---|---|
| SEQ ID NO. | Oligonucleotide | Sequence (5′→3′) |

| | | |
|---|---|---|
| 1 | 12W^{a} | GTTGGAGCTGGTGGCGTAGGC |
| 2 | 12M^{b}1 | AGTTGGAGCTTGTGGCGTAGG |
| 3 | 12M2 | AGTTGGAGCTAGTGGCGTAGG |
| 4 | 12M3 | AGTTGGAGCTCGTGGCGTAGG |
| 5 | 12M4 | GTTGGAGCTGATGGCGTAGGC |
| 6 | 12M5 | GTTGGAGCTGCTGGCGTAGGC |
| 7 | 12M6 | GTTGGAGCTGTTGGCGTAGGC |
| 8 | 12M7 | TTGGAGCTGGAGGCGTAGGCA |
| 9 | 12M8 | TTGGAGCTGGGGGCGTAGGCA |
| 10 | 12M9 | TTGGAGCTGGCGGCGTAGGCA |
| 11 | 13W | GGAGCTGGTGGCGTAGGCAAG |
| 12 | 13M1 | TGGAGCTGGTCGCGTAGGCAA |
| 13 | 13M2 | TGGAGCTGGTAGCGTAGGCAA |
| 14 | 13M3 | TGGAGCTGGTTGCGTAGGCAA |
| 15 | 13M4 | GGAGCTGGTGCCGTAGGCAAG |
| 16 | 13M5 | GGAGCTGGTGACGTAGGCAAG |
| 17 | 13M6 | GGAGCTGGTGTCGTAGGCAAG |
| 18 | 13M7 | GAGCTGGTGGTGTAGGCAAGA |
| 19 | 13M8 | GAGCTGGTGGAGTAGGCAAGA |
| 20 | 13M9 | GAGCTGGTGGGGTAGGCAAGA |

^{a}W: wild-type,
^{b}M: mutant type

All 20 oligonucleotides, each having a 12-carbon spacer to 5′-terminal modified with an amine residue which can undergo Schiff's base reaction with aldehyde groups, were obtained from Metabion (Germany) and purified by HPLC.

The K-ras oligonucleotide microarray of the present invention was manufactured as previously described (Kim I J, et al., Hardiman G ed. Microarrays methods and applications—nuts & bolts. Eagleville, DNA press, 249-72, 2003). In particular, each oligonucleotide was mixed with a micro spotting solution (TeleChem International Inc, Sunnyvale, Calif.) at a mix ratio of 1:1, and 40 μl of each oligonucleotide was transferred to a 96 well plate. Forty pmol/μl of oligonucleotides were spotted for codons 12 and 13. After the charged 96 well plate was placed in a pin microarrayer (Microsys 5100 Cartesian, Cartesian Technologies Inc, Irvine, Calif.), each oligonucleotide was printed on an aldehyde-coated glass slide (26×76×1 mm, CEL Associates Inc, Houston, Tex.). Spots, each of 130 μm diameter in size, were arranged in multiple columns and rows at intervals of 300 μm. A total of 80 (20×4) oligonucleotides were arrayed in a quadruplicate manner, which consisted of 2 wild-types and 18 missense mutation types covering codons 12 and 13 of the K-ras gene. Three oligonucleotide sets were spotted separately on one slide, such that 3 different samples could hybridize with one microarray.

The glass slide spotted with the oligonucleotides was washed twice with 0.2% SDS, and then, once with distilled water. The glass slide was soaked in hot water (95° C.) to denature the oligonucleotides, and then, in a sodium borohydride solution for 5 minutes to inactivate unreacted aldehyde groups. Then, the glass slide was washed twice with 0.2% SDS, and then, once with distilled water, centrifuged, and dried.

### EXAMPLE 2

### Examination of K-ras Mutation using K-ras Oligonucleotide Microarray

(Step 1) Preparation of DNA sample

A total of 204 colorectal cancer patients from Seoul National University Hospital and National Cancer Center of Korea were investigated for the presence of somatic K-ras mutation. Written informed consents were obtained from all patients. Of the 204 colorectal cancers, 103 were from the proximal colon (cecum to splenic flexure) and 101 were from the distal colorectum (splenic flexure to rectum). Further, a normal tissue of colorectal cancer patient was used as a negative control.

Genomic DNA was extracted from frozen specimens using TRI reagent (Molecular Research Center, Cincinnati, Ohio, USA) as previously described (Kim I J, et al., Clin. Cancer Res. 9: 2920-2925, 2003). To generate a fluorescent dye-labeled DNA sample, PCR amplification was performed using the extracted DNA as a template and two pairs of primers of SEQ ID NOs. 21 and 22 (Metabion, Germany) as previously described (Kim I J, et al., Clin. Cancer Res. 8: 457-463, 2002; Kim, I J, et al., Clin. Cancer Res. 9: 2920-2925, 2003).

A PCR reaction solution (25 μl) contained 100 ng of genomic DNA, 10 pmol of each primer, 50 μM each of dATP, dTTP and dGTP (MBI Fermentas), 10 μM each of fluorescent dye labeled Cy5-dCTP (Amersham Bioscience) and dCTP. Reactions were initiated by denaturation for 5 min at 94° C. in a programmable thermal cycler (Perkin Elmer Cetus 9600; Roche Molecular Systems, Inc., NJ). The PCR condition consisted of 35 cycles of 30 sec at 94° C., 30 sec at 56° C., and 1 min at 72° C., with a final elongation of 7 min at 72° C.

For the CDH method, DNA samples were labeled with an additional fluorescent dye: Cy3-dCTP (Amersharm Bioscience) or Cromatide™-dUTP-Alexa fluor 594 (Molecular Probes). In PCR reaction for CDH, each sample was amplified with those fluorescent labeled-dNTPs and these were incorporated with DNA.

After the PCR amplification, each of Cy5-, Cy3- and Alexa™ 594-labeled PCR products was purified using a purification kit (QIAquick PCR purification kit, Qiagen Inc, Valencia, Calif.) and digested with 0.05 U of DNase I (Takara, Shiga, Japan) at 25° C. for 3 min. Remaining enzyme was inactivated at 80° C. for 10 min, and the Cy5-, Cy3- and Alexa™ 594-labeled DNA samples were each recovered.

(Step 2) Hybridization Reaction and Analysis

The Cy5-, Cy3- and Alexa™ 594-labeled DNA samples prepared in step (1) were mixed and resuspended in 5× hybridization solution (Hybit, TeleChem International Inc, Sunnyvale, Calif.) to a volume of 2˜4 μl. Two μl of the mixed DNA sample was dropped on the glass slide manufactured in Example 1 and the glass slide was covered with a cover glass. The hybridization reaction was performed by incubating the glass slide in a saturated vapor tube at 56° C. for 2.5 hours. This procedure made DNA samples, each amplified from patients and having a specific tag, compete with each other in the hybridization reaction within the limited amount of spotted oligonucleotide.

The hybridized glass slide was rinsed at room temperature in a buffer of 0.2% SDS+0.5×SSC for 15˜30 min, and then, in distilled water for 5 min, followed by centrifuging and drying. The glass slide was scanned to calculate the intensity of each spot, which represented the amount of hybridized DNA from tumor, by image analysis ScanArray Lite (Parkard Instrument Co, Meriden, Conn.) and QuantArray (version 2.0, Parkard Instrument Co, Meriden, Conn.). According to each fluorescent dye's excitation wavelength, hybridized microarray was scanned at wavelengths of 632.8 nm, 543.8 nm and 594 nm for Cy5, Cy3 and Alexa™ 594, respectively (Lovmar L, et al., Nucleic Acids Res. 31: e129, 2003).

Two wild type signals were compared with each other and adjusted to be equal by signal normalization. The remaining 18 signals at each codon were also adjusted in the same way as the wild type signals. After signal normalization, all signals were re-analyzed as previously described (Kim, I J, et al., Clin. Cancer Res. 8: 457-463, 2002). The mean (BA) and the standard deviation (BSD) of the background signals were calculated, and the cutoff level was established to be BA+2.58BSD. (BA+2.58BSD) indicated the upper limit of the 99% confidence interval, and signals over this value were identified as meaningful signals. All data analysis was carried out using a SigmaPlot (SPSS Inc., San Rafael, Calif.), and means and standard deviations were calculated using Microsoft Excel program.

As a result, a total of 50 mutations were identified in colorectal cancers ( 50/204, 24.5%) by K-ras oligonucleotide microarray. Of these, 28 were from proximal colon cancers ( 28/103, 27.2%) and 22 were from distal colorectal cancers ( 22/101, 21.8%). A total of four types of missense mutation causing amino acid change in codon 12 or 13 were detected. The most common types of mutation were GGC (Gly)GAC (Asp, 21/50) in codon 13. Others were changed from GGT (Gly) to GAT (Asp, 16/50), GTT (Val, 8/50), and TGT (Cys, 5/50). However, there was no detected in a normal tissue of cancer patient.

FIGS. 1a to 1e showed scanned images and each of signal intensity of K-ras oligonucleotide microarray with (CDH group) or without applying the CDH method (control group). FIG. 1a was the result of conventionally hybridizing with D231 sample amplified with Cy5-labeled dCTP (D231-control); FIG. 1b, competitively hybridizing with D231 sample amplified with Cy5-, Cy3- and Alexa™ 594-labeled dCTP (D231-CDH); FIG. 1c, conventionally hybridizing with D281 sample amplified with Cy3-labeled dCTP (D281-control); and FIG. 1d, competitively hybridizing with D231 sample amplified with Cy5-, Cy3- and Alexa™ 594-labeled dCTP (D281-CDH). A missense mutation at codon 13 (GGC→GAC) was detected in D231 sample by using Cy5-labeled dCTP, and a missense mutation at codon 12 (GGT→GAT) was detected in D281 sample by using Cy3-labeled dCTP. FIG. 1e was the result of competitively hybridizing with a normal tissue of cancer patient (negative control), and there was no detected K-ras mutation. The mutation was indicated by arrow, and signal intensities of spotted oligonucleotide were plotted after normalization based on the wild-type's signal. Some of non-specific bindings were also detected (*). Comparing CDH (FIGS. 1b and 1d) with its control (FIG. 1a and 1c), it was found that signals of non-specific binding decreased and the ratio of mutation and wild-type (R) increased (D231; 0.91→1.66, D281; 0.28→0.56). It was also detected that two signals of the wild-type (codons 12 and 13) were somewhat reduced. The reason was that fragmented wild-type DNAs from each sample participated in the hybridization. But, mutant DNAs, rarely having a common sequence in the state of three mixed samples, did not compete and conserved its original signal.

In order to investigate any significance between the mutation profile and phenotype, statistical analyses were performed using the χ² or Fisher's exact test with SPSS software. α=0.05 was set as the significance level. As a result, it was found that GGT→GAT type is more prevalent in proximal colon cancer ( 13/28) than in distal colon cancer ( 3/22, p=0.014), which agrees in concordance with previous reports (Samowitz W S, e al., Cancer Epidemiol. Biomarkers Prev. 9: 1193-1197, 2000; Brink M, et al., Carcinogenesis 24: 703-710, 2003). However, no significant relationship was detected between K-ras mutation and sex, age, tumor size, differentiation, and TNM stage.

### EXAMPLE 3

### Confirmation of K-ras Mutations Detected by K-ras Oligonucleotide Microarray

In order to confirm K-ras mutations detected by the K-ras oligonucleotide microarray of the present invention, 205 colorectal cancer samples were subjected to bi-directional sequencing analysis as previously described (Park, J H, et al., Clin. Genet. 64: 48-53, 2003). For sequencing, previously reported primers of SEQ ID NOs: 23 and 24 were used (Lagarda H, et al., J. Pathol. 193: 193-199, 2001). PCR was performed according to the same method as described in the step (1) of Example 2, except for using a conventional dNTP mixture.

Bi-directional sequencing was performed using a Taq dideoxy terminator cycle sequencing kit and an ABI 3100 DNA sequencer (Applied Biosystems, Forster City, Calif.).

As a result, mutation results were 100% concordant with direct sequencing, showing neither false-positive nor false-negative.

While the embodiments of the subject invention have been described and illustrated, it is obvious that various changes and modifications can be made therein without departing from the spirit of the present invention which should be limited only by the scope of the appended claims.

What is claimed is:

1. A K-ras oligonucleotide microarray for detecting K-ras mutations comprising a plurality of oligonucleotides fixed on the surface of a solid matrix, wherein the oligonucleotides are designed to detect missense mutation types at mutational hot spots of K-ras gene and comprise a wild-type having the nucleotide sequence of SEQ ID NO. 1 and missense mutation types having the nucleotide sequences of SEQ ID NOs: 2 to 10 at codon 12; and a wild-type having the nucleotide sequence of SEQ ID NO. 11 and missense mutation types having the nucleotide sequences of SEQ ID NOs: 12 to 20 at codon 13.

2. The K-ras oligonucleotide microarray of claim 1, wherein each of the oligonucleotides has a 12 carbon spacer with 5′ amino modification, and the solid matrix is coated with an aldehyde or amine.

3. The K-ras oligonucleotide microarray of claim 2, wherein the oligonucleotides are fixed on the solid matrix surface by way of forming covalent bonds between the amine groups of the oligonucleotides and the aldehyde groups of the solid matrix via Schiff's base reaction.

4. A method for detecting K-ras mutation using the K-ras oligonucleotide microarray of claim 1, comprising
1) preparing a fluorescent dye-labeled DNA;
2) reacting the labeled DNA sample with oligonucleotide spots on the K-ras oligonucleotide microarray;
3) washing the reacted microarray to remove unbound sample DNA;
4) detecting the mode of hybridization of specific oligonucleotide spots using a fluorescence reader; and
5) examining the presence of gene mutation.

5. The method of claim 4, wherein the sample is a tumor specimen or a blood obtained from a target patient.

6. The method of claim 4, wherein the hybridization reaction is carried out according to a competitive DNA hybridization (CDH) method.

7. The method of claim 4, wherein the competitive DNA hybridization method comprises the steps of: mixing at least two samples amplified with different fluorescent dye-labeled dNTPs; dropping the sample mixture in one spotted oligonucleotide on the surface of the microarray; and making the samples compete with each other in the hybridization reaction within the limited amount of spotted oligonucleotide.

8. The method of claim 7, wherein the fluorescent dye is selected from the group consisting of Cy5, Cy3, Alexa fluor, Texas Red, Fluorescein and Lissamine.

9. The method of claim 4, wherein the hybridization reaction is performed in a 45˜60° C. incubator saturated with water vapor for 3˜9 hours.
